(19) United States
(12) Patent Application Publication
(10) Pub. No.: US 2013/0041281 A1
(43) Pub. Date: Feb. 14, 2013

(54) APPARATUS AND METHOD FOR GENERATING BRAIN WAVES FOR SUPPRESSING APPETITE WHILE DIETING

(71) Applicants: Un Hee PARK, Gwangju (KR); Dae Sub Oh, Daejeon (KR); Bon Jun Ku, Daejeon (KR); Do Seob Ahn, Daejeon (KR)

(72) Inventors: Un Hee PARK, Gwangju (KR); Dae Sub Oh, Daejeon (KR); Bon Jun Ku, Daejeon (KR); Do Seob Ahn, Daejeon (KR)

(73) Assignee: Electronics and Telecommunications Research Institute, Daejeon (KR)

(21) Appl. No.: 13/549,161

(22) Filed: Jul. 13, 2012

(30) Foreign Application Priority Data
Aug. 12, 2011 (KR) 10-2011-0080384
Mar. 9, 2012 (KR) 10-2012-0024499

(51) Int. Cl. A61B 5/0482

(52) U.S. Cl. 600/545

(57) ABSTRACT

An apparatus and method for generating brain waves for dieting of a user are disclosed. The brain wave generating apparatus may include a brain wave detection unit to detect whether brain waves related to an increase in appetite are generated from a user; and a brain wave generation unit to generate brain waves related to suppression of appetite when the brain waves related to the increase in appetite are generated from the user.

### CROSS-REFERENCE TO RELATED APPLICATIONS

This application claims the benefit of Korean Patent Application No. 10-2011-0080384 and of Korean Patent Application No. 10-2012-0024499, respectively filed on Aug. 12, 2011 and Mar. 9, 2012, in the Korean Intellectual Property Office, the disclosures of which are incorporated herein by reference.

### BACKGROUND

1. Field of the Invention

The present invention relates to an apparatus and method for generating brain waves to assist a user with dieting, and more particularly, to an apparatus and method for dieting that generates brain waves for suppressing an appetite of the user, by introducing the brain waves that induce the user feel full when the user who is dieting feels hungry.

2. Description of the Related Art

Due to dietary habits and changes in diets of modern people, an obese populace is gradually increasing and, accordingly, a population of dieters is also increasing.

However, a difficulty most commonly experienced by dieters is suppressing an appetite. Although various pharmaceuticals which assist in suppressing the appetite have been developed, most of these chemicals have a side effect that results in that organs, such as a liver, may be distressed. Therefore, prescriptions from a doctor are required for these pharmaceuticals. Further, dieters are usually prevented from ingesting these pharmaceuticals continuously for more than a predetermined period of time.

Accordingly, there is a desire for a new safe method for suppressing an appetite of a user without a need for use of pharmaceuticals.

### SUMMARY

An aspect of the present invention provides an apparatus and method for suppressing an appetite of a user who is on a diet, using brain waves.

According to an aspect of the present invention, there is provided a brain wave generating apparatus including a brain wave detection unit to detect whether brain waves related to an increase in appetite are generated from a user; and a brain wave generation unit to generate brain waves related to suppression of appetite when the brain waves related to the increase in appetite are generated from the user.

The brain wave generating apparatus may further include a brain wave transmission unit to transmit the brain waves related to suppression of appetite to a brain introduction apparatus.

The brain wave generating apparatus may further include an appetite reduction confirmation unit to confirm whether an appetite of the user is reduced after the brain waves related to the suppression of appetite are introduced to the user.

According to another aspect of the present invention, there is provided a brain wave generating method including detecting whether brain waves related to an increase in appetite are generated from a user; and generating brain waves related to suppression of appetite when the brain waves related to the increase in appetite are generated from the user.

### Effect

According to embodiments of the present invention, when a user who is on a diet feels hungry, brain waves may be introduced to the user so that the user may feel full, thereby suppressing the appetite of the user without a need for use of pharmaceuticals.

### BRIEF DESCRIPTION OF THE DRAWINGS

These and/or other aspects, features, and advantages of the invention will become apparent and more readily appreciated from the following description of exemplary embodiments, taken in conjunction with the accompanying drawings of which:

FIG. 1 is a diagram illustrating relationships between a brain wave generating apparatus for dieting and another apparatus according to an embodiment of the present invention; FIG. 2 is a block diagram illustrating a brain wave generating apparatus for dieting according to an embodiment of the present invention;

FIG. 3 is a flowchart illustrating a brain wave generation method for dieting according to an embodiment of the present invention; and

FIG. 4 is a flowchart illustrating a state change of a user according to a brain wave generation method for dieting according to an embodiment of the present invention.

### DETAILED DESCRIPTION

Reference will now be made in detail to exemplary embodiments of the present invention, examples of which are illustrated in the accompanying drawings, wherein like reference numerals refer to the like elements throughout. Exemplary embodiments are described below to explain the present invention by referring to the figures. A brain wave generating method for dieting according to an embodiment of the present invention may be performed by a brain wave generating apparatus 100 for dieting. When a ventromedial nucleus of the hypothalamus of a user is stimulated, the user loses an appetite. When the user eats food, hormones, such as peptide YY (PYY) or glucagon-like peptide (GLP-1), that make the user feel full and satisfied are generated by internal organs of the user. The generated hormones are transferred to a brain of the user, thereby inducing the user to stop eating.

That is, the brain wave generating apparatus 100 may generate appetite suppressing brain waves capable of controlling an appetite control function of the hypothalamus and introduce the appetite suppressing brain waves into the user, thereby suppressing the appetite of the user.

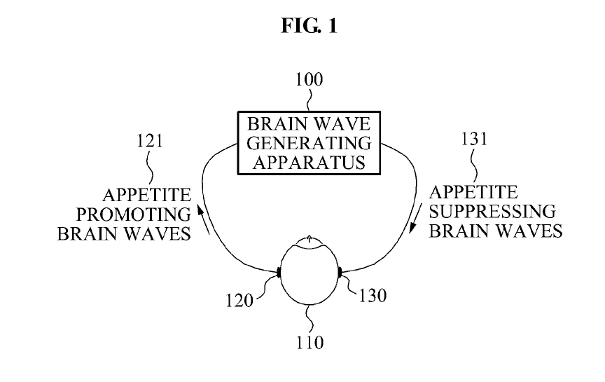

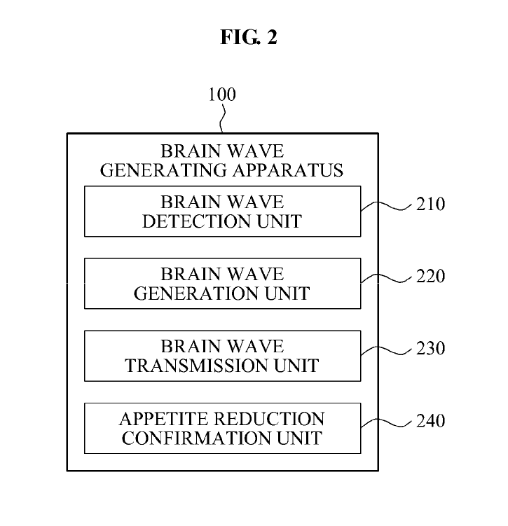

FIG. 1 illustrates relationships between the brain wave generating apparatus 100 for dieting according to the embodiment of the present invention with another apparatus.

When receiving appetite promoting brain waves 121, that is, brain waves related to an increase in appetite, from a brain wave sensor 120 attached to a head 110 of the user as shown in FIG. 1, the brain wave generating apparatus 100 may determine that the user feels hungry and generate appetite suppressing brain waves 131, that is, brain waves related to suppression of appetite.

Here, the appetite promoting brain waves 121 may be brain waves generated when the user feels hungry or feels like eating. The appetite suppressing brain waves 131 may be brain waves requesting generation of a substance making the user feel full.

The brain wave generating apparatus 100 may transmit the appetite suppressing brain waves 131 to a brain wave introducing apparatus 130 attached to the head 110 of the user. The brain wave introducing apparatus 130 may introduce the received appetite suppressing brain waves 131 into the user. That is, the brain wave introducing apparatus 130 may transmit the received appetite suppressing brain waves 131 to a brain of the user. The substance making the user feel full is generated from the user by the appetite suppressing brain waves 131 being introduced. Therefore, the user may feel full and accordingly lose the appetite or easily suppress the appetite.

That is, the brain wave generating apparatus 100 may suppress an appetite of the user who is on a diet, by introducing the brain waves that make the user feel full when the user feels hungry.

FIG. 2 illustrates a brain wave generating apparatus 100 according to an embodiment of the present invention.

Referring to FIG. 2, the brain wave generating apparatus 100 may include a brain wave detection unit 210, a brain wave generation unit 220, a brain wave transmission unit 230, and an appetite reduction confirmation unit 240.

The brain wave detection unit 210 may detect whether appetite promoting brain waves are generated from the user. That is, the brain wave detection unit 210 may receive brain waves of the user from a brain wave sensor 120 attached to a head of the user, and confirm whether the appetite promoting brain waves are included in the received brain waves. Here, when the brain waves of the user include the appetite promoting brain waves, the brain wave detection unit 210 may request generation of appetite suppressing brain waves from the brain wave generation unit 220.

The appetite promoting brain waves may include at least one selected from brain waves stimulating a lateral hypothalamus, brain waves reducing serotonin which is an appetite suppressing neurotransmitter, and brain waves increasing ghrelin which is an appetite promoting hormone.

The brain wave generation unit 220 may generate the appetite suppressing brain waves according to the request by the brain wave detection unit 210.

Here, the appetite suppressing brain waves may include at least one selected from brain waves inducing secretion of an appetite suppressing hormone, such as leptin or a peptide, brain waves increasing serotonin which is the appetite suppressing neurotransmitter, and brain waves reducing ghrelin which is the appetite promoting hormone.

The brain wave transmission unit 230 may introduce the appetite suppressing brain waves generated by the brain wave generation unit 220 into the user, by transmitting the appetite suppressing brain waves to a brain wave introducing apparatus 130.

The appetite reduction confirmation unit 240 may confirm whether the appetite of the user is reduced after the brain wave transmission unit 230 introduces the appetite suppressing brain waves into the user through the brain wave introducing apparatus 130.

The appetite reduction confirmation unit 240 may detect through the brain wave detection unit 210 whether brain waves related to fullness or satisfaction are generated from the user, and determine the appetite of the user to be reduced when the brain waves related to fullness or satisfaction are generated from the user.

When the brain waves related to fullness or satisfaction are not generated from the user, the appetite reduction confirmation unit 240 may request transmission of the appetite suppressing brain waves from the brain wave transmission unit 230 so that the appetite suppressing brain waves are continuously introduced into the user until the appetite of the user is suppressed.

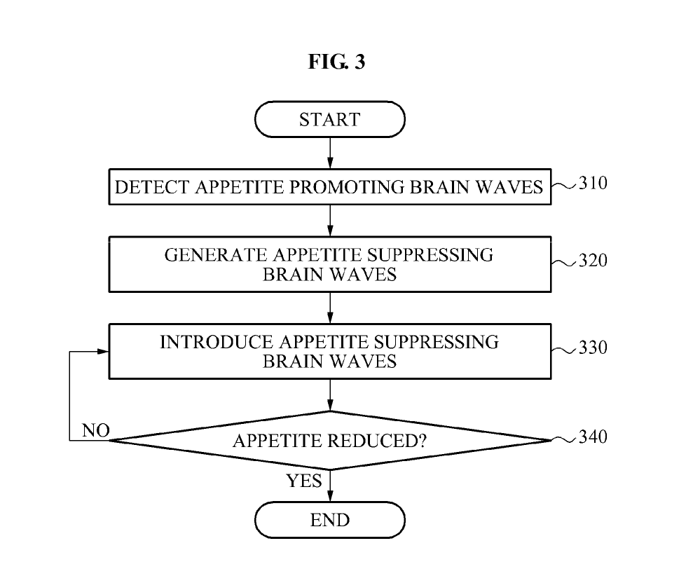

FIG. 3 illustrates a brain wave generating method for dieting, according to an embodiment of the present invention.

In operation 310, the brain wave detection unit 210 may detect whether the appetite promoting brain waves are generated from the user. That is, the brain wave detection unit 210 may receive brain waves of the user from the brain wave detection sensor 120 attached to the head of the user and confirm whether the appetite promoting brain waves are included in the received brain waves.

When the appetite promoting brain waves are detected in operation 310, the the brain wave generation unit 110 may generate the appetite suppressing brain waves for suppressing the appetite of the user in operation 320.

In operation 330, the brain wave transmission unit 230 may transmit the appetite suppressing brain waves generated in operation 320 to the brain wave introducing apparatus 130, thereby introducing the appetite suppressing brain waves into the user.

In operation 340, the appetite reduction confirmation unit 240 may confirm whether the appetite of the user is reduced due to the appetite suppressing brain waves introduced in operation 330. Here, the appetite reduction confirmation unit 240 may detect through the brain wave detection unit 210 whether the brain waves related to fullness or satisfaction are generated from the user, and determine the appetite of the user to be reduced when the brain waves related to fullness or satisfaction are generated from the user.

When the brain waves related to fullness or satisfaction are not generated from the user, the appetite reduction confirmation unit 240 may perform operation 330 once again so that the appetite suppressing brain waves are continuously introduced into the user until the appetite of the user is suppressed.

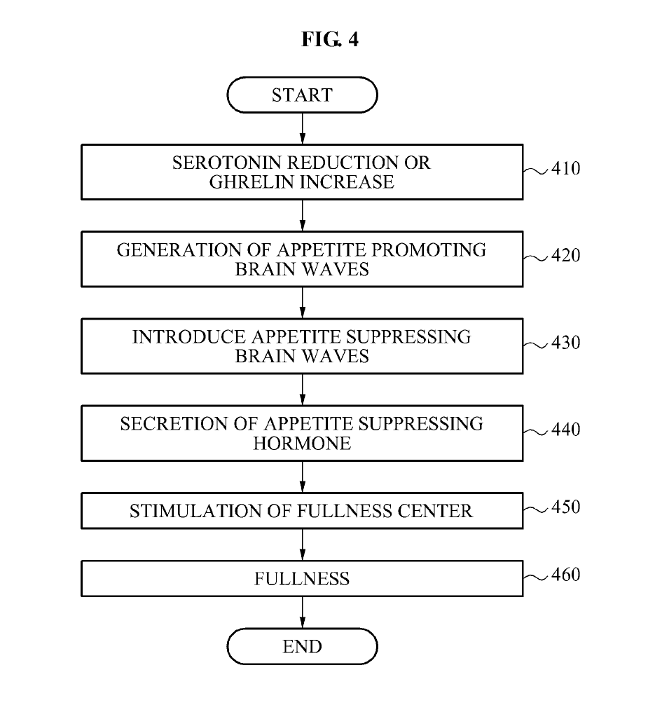

FIG. 4 illustrates a state change of a user according to a brain wave generation method for dieting according to an embodiment of the present invention.

In operation 410, when the user feels stressed or hungry, a lateral hypothalamus of the user may be stimulated and, therefore, serotonin which is the appetite suppressing neurotransmitter may be reduced or Ghrelin which is the appetite promoting hormone may be increased.

In operation 420, the user may feel hungry as a level of serotonin reduces or a level of gherelin increases in operation 410. In this instance, the appetite promoting brain waves may be generated from the brain of the user. Therefore, the brain wave generating apparatus 100 detecting the appetite promoting brain waves may generate the appetite suppressing brain waves.

In operation 430, the user may receive the appetite suppressing brain waves from the brain wave generating apparatus 100.

In operation 440, the user may secrete the appetite suppressing hormone such as leptin or a peptide by the brain waves received in operation 430.

In operation 450, the user may secrete peptide YY (PYY) or glucagon-like peptide (GLP-1) which is a chemical substance that stimulates the fullness center of the user and thereby makes the user feel full, due to the appetite suppressing hormone secreted in operation 440.

In operation 460, the user may feel full due to the peptide YY (PYY) or the glucagon-like peptide (GLP-1) secreted in operation 450 and lose the appetite.

According to the embodiments of the present invention, when the user who is on a diet feels like eating, brain waves that make the user feel full are introduced into the user. Therefore, the user may suppress the appetite without a need for using dedicated pharmaceuticals.

Although a few exemplary embodiments of the present invention have been shown and described, the present invention is not limited to the described exemplary embodiments. Instead, it would be appreciated by those skilled in the art that changes may be made to these exemplary embodiments without departing from the principles and spirit of the invention, the scope of which is defined by the claims and their equivalents.

What is claimed is:

1. A brain wave generating apparatus comprising:
a brain wave detection unit to detect whether brain waves related to an increase in appetite are generated from a user; and
a brain wave generation unit to generate brain waves related to suppression of appetite when the brain waves related to the increase in appetite are generated from the user.

2. The brain wave generating apparatus of claim 1, wherein the brain waves related to the increase in appetite are brain waves that reduce an appetite suppressing neurotransmitter.

3. The brain wave generating apparatus of claim 1, wherein the brain waves related to the increase in appetite are brain waves that increase an appetite promoting hormone.

4. The brain wave generating apparatus of claim 1, wherein the brain waves related to suppression of appetite are brain waves that induce secretion of an appetite suppressing hormone.

5. The brain wave generating apparatus of claim 1, wherein the brain waves related to the suppression of appetite are brain waves that increase an appetite suppressing neurotransmitter.

6. The brain wave generating apparatus of claim 1, wherein the brain waves related to the suppression of appetite are brain waves that reduce an appetite promoting hormone.

7. The brain wave generating apparatus of claim 1, further comprising a brain wave transmission unit to transmit the brain waves related to suppression of appetite to a brain introduction apparatus,
wherein the brain wave introduction apparatus introduces the brain waves related to the suppression of appetite into the user.

8. The brain wave generating apparatus of claim 7, further comprising:
an appetite reduction confirmation unit to confirm whether an appetite of the user is reduced after the brain waves related to the suppression of appetite are introduced into the user.

9. The brain wave generating apparatus of claim 8, wherein the appetite reduction confirmation unit detects whether brain waves related to fullness or satisfaction are generated from the user, and determines the appetite of the user to be reduced when the brain waves related to fullness or satisfaction are generated from the user.

10. A brain wave generating method comprising:
detecting whether brain waves related to an increase in appetite are generated from a user; and
generating brain waves related to suppression of appetite when the brain waves related to the increase in appetite are generated from the user.

11. The brain wave generating method of claim 10, wherein the brain waves related to the increase in appetite are brain waves that reduce an appetite suppressing neurotransmitter.

12. The brain wave generating method of claim 10, wherein the brain waves related to the increase in appetite are brain waves that increase an appetite promoting hormone.

13. The brain wave generating method of claim 10, wherein the brain waves related to suppression of appetite are brain waves that induce secretion of an appetite suppressing hormone.

14. The brain wave generating method of claim 10, wherein the brain waves related to the suppression of appetite are brain waves that increase an appetite suppressing neurotransmitter.

15. The brain wave generating method of claim 10, wherein the brain waves related to the suppression of appetite are brain waves that reduce an appetite promoting hormone.

16. The brain wave generating method of claim 10, further comprising introducing the brain waves related to suppression of appetite to the user.

17. The brain wave generating method of claim 16, further comprising confirming whether an appetite of the user is reduced after the brain waves related to the suppression of appetite are introduced to the user.

18. The brain wave generating apparatus of claim 17, wherein the confirming comprises:
detecting whether brain waves related to fullness or satisfaction are generated from the user; and
determining the appetite of the user to be reduced when the brain waves related to fullness or satisfaction are generated from the user.
